(19) United States
(12) Patent Application Publication
(10) Pub. No.: US 2007/0191741 A1
(43) Pub. Date: Aug. 16, 2007

(54) Image analysis methods for gleno-humeral joint morphology

(75) Inventors: Ming-Dar Tsai, Taipei City (TW); Ming-Shium Hsieh, Taipei City (TW)

Correspondence Address: QUINTERO LAW OFFICE, PC, 2210 MAIN STREET, SUITE 200, SANTA MONICA, CA, 90405, US

(73) Assignees: CHUNG YUAN CHRISTIAN UNIVERSITY, TAOYUAN COUNTRY (TW); TAIPEI MEDICAL UNIVERSITY, TAIPEI CITY (TW)

(21) Appl. No.: 11/489,798

(22) Filed: Jul. 19, 2006

(30) Foreign Application Priority Data
Feb. 15, 2006 (TW) TW095105025

(51) Int. Cl. A61B 5/107; A61B 17/88

(52) U.S. Cl. 600/587; 623/908; 606/102

(57) ABSTRACT

Image analysis methods for gleno-humeral joint morphology. At least one specific structure is approximated as an elliptical structure at a plurality of transverse sections. At least one pathological feature on the structure is recognized. At least one structural spatial property of a 3D structure is calculated based on the structural property of the elliptical structure, thus determining the morphology of the 3D structure. Structural deformities are evaluated according to the morphology of the 3D structure. The pathological features on the sections are integrated to obtain at least one 3D pathological feature property, and a treatment is determined accordingly.

### BACKGROUND OF THE INVENTION

1. Field of the Invention

The present disclosure relates generally to image analysis methods for gleno-humeral (GH) joint morphology, and, more particularly to image analysis methods that automate the GH joint diagnoses and surgical management using computed tomography (CT) transverse sections.

2. Description of the Related Art

FIG. 1A is a schematic diagram illustrating an idealized GH joint, at which the contact joint of the humeral head rotates inside the glenoid cavity to transfer the load on the humeral stem to the glenoid (G). The ratios, distances and angles among the contact joint, the stem and the glenoid must be in normal ranges to maintain GH joint stability. Some conditions can change the ratios, angles or distances to result in abnormalities in rotation or stability. For example, a dislocated humerus results in instability because of large deviation from the contact joint center to the ideal position (on the glenoid attitude vector (GAV)) such that it cannot rotate stably inside the glenoid cavity as shown in FIG. 1B, and a fractured segment (FF) obstructs the humerus rotation as shown in FIG. 1C.

A set of CT or MRI (Magnetic Resonance Imaging) transverse sections may be used to resolve the humerus and glenoid. The most inferior sections (Section A in FIG. 1A) resolve only the humeral stem. More superior sections resolve the tubercle or the contact joint or the glenoid depending on the section position and the arm attitude. For example, Section B in FIG. 1A resolves only the tubercle, Section C resolves the contact joint and the tubercle and Section E resolves the contact joint and the glenoid, while Section D resolves all the tubercle, the contact joint and the glenoid.

To facilitate the GH joint function, the largest possible prosthesis is used to reduce average load, fit the prosthetic stem axis to the stem canal and produce a suitable humeral or prosthetic head position to stabilize GH joint motion. Additionally, procedures should automatically select the cutting plane for inserting the prosthesis, positions for screws and plates or nails and prosthetic components.

Recently, computer graphics techniques have enabled real-time visualization and interactive surgical simulation for CT or MRI sections to assist diagnosis and surgical management. To achieve this purpose, feature recognition techniques for intervertebral discs, spinal bones and hip structures based on 2D transverse CT or MRI sections have been developed. The image analysis results on these 2D sections are then integrated to evaluate 3D structure morphological properties and thus obtain spatial pathological characteristics to automate precise diagnosis and surgical management for diseases of the intervertebral discs, spinal bones and hip. The managed surgical modalities can be simulated by a surgery simulator. This orthopedic simulator accurately represents the surface topology and geometry of an anatomic structure to enable the closure check for the intersections of swept surfaces of surgical tools with the anatomic structure. Thus, this simulator can recognize new bones generated from the cut swept surfaces on bones to enable various orthopedic surgical procedures such as removal, repositioning and fusion. The simulation results of every procedure in a surgical modality can demonstrate how bones are opened, corrected or repositioned, closed and fused, how a prosthesis is inserted, and how screws and plate are positioned.

### BRIEF SUMMARY OF THE INVENTION

Image analysis methods for gleno-humeral joint morphology are provided.

In an embodiment of an image analysis method for gleno-humeral joint morphology, at least one specific structure is approximated as an elliptical structure at a plurality of transverse sections. At least one pathological feature on the structure is recognized. At least one structure spatial property of a 3D structure is calculated based on the structure property of the elliptical structure, thus determining the morphology of the 3D structure. Structural deformities of the 3D structure are evaluated according to the morphology of the 3D structure. The pathological features on the sections are integrated to obtain at least one 3D pathological feature property, and a treatment is determined accordingly.

Image analysis methods for gleno-humeral joint morphology may take the form of program code embodied in a tangible media. When the program code is loaded into and executed by a machine, the machine becomes an apparatus for practicing the disclosed method.

### BRIEF DESCRIPTION OF THE DRAWINGS

The invention will become more fully understood by referring to the following detailed description with reference to the accompanying drawings, wherein:

FIG. 1A is a schematic diagram illustrating an idealized GH joint;

FIG. 1B is a schematic diagram illustrating a dislocated humerus;

FIG. 1C is a schematic diagram illustrating a fractured segment obstructing humerus rotation;

FIGS. 2A, 2B and 2C are schematic diagrams illustrating structure and feature recognition on transverse sections;

FIGS. 3A, 3B and 3C illustrate 3D structure and feature calculation;

FIGS. 4A, 4B and 4C are images of isosurface reconstruction and analysis for a humeral head fracture and dislocation;

FIGS. 5A, 5B and 5C are illustrate surgical simulations for reduction of head fracture and dislocation;

FIGS. 6A, 6B and 6C are images of isosurface reconstruction and analysis for a head tumor and fracture;

FIGS. 7A, 7B and 7C illustrate surgical simulations for arthroplasty;

FIGS. 8A, 8B and 8C are images of isosurface reconstruction, analysis and surgical simulation for an avulsion; and

FIG. 9 is a flowchart of an embodiment of an image analysis method for gleno-humeral joint morphology.

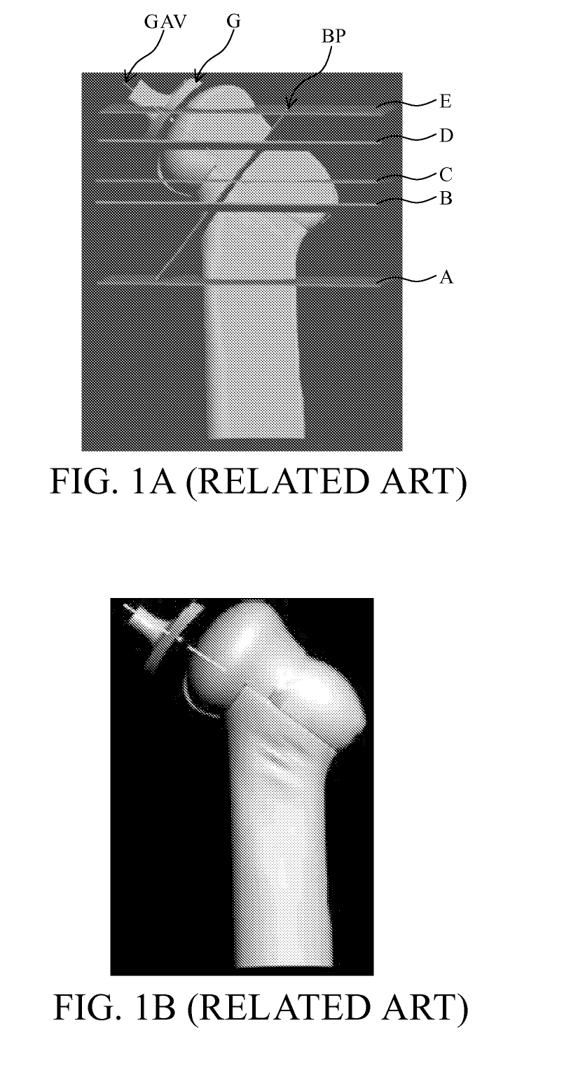

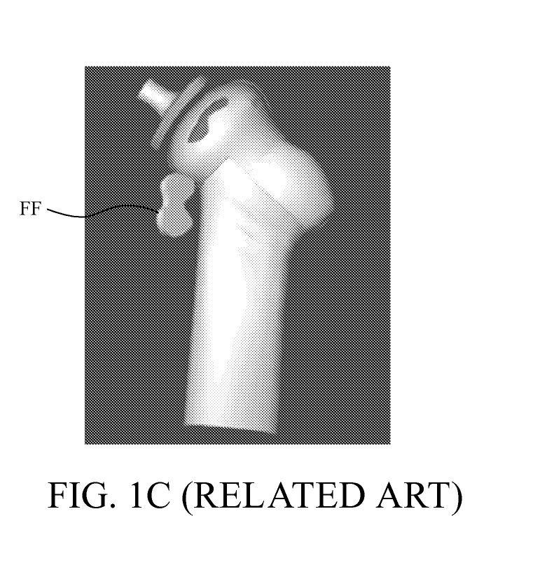

### DETAILED DESCRIPTION OF THE INVENTION

Image analysis methods for gleno-humeral joint morphology are provided.

In the invention, methods that use successive transverse CT sections to evaluate the humerus and glenoid morphology for automatic GH joint diagnoses and surgery managements are provided. The methods identify the humeral stem, tubercle and contact joint as well as the glenoid to recognize concave, convex, and hole features on these structures. Such features on the successive sections are integrated to indicate abnormalities of spurs, fractures and tumors and their position and volume. The structural properties of the sections such as radius of the contact joint, the glenoid, or the humeral stem are then used to calculate the structural spatial properties such as the contact joint center and radius, the glenoid attitude, the boundary plane (BP) between the contact joint and tubercle, and the stem axis. These properties can be used to evaluate whether a structure is dislocated or compressed, or the humeral stem axis is sheared. Based on these structure and feature evaluations, surgical procedures are then automatically managed to dissect tumors and bone grafts, reduce the dislocated humerus and compressed structures, or position a prosthesis or screws and plate. These surgical procedures are then simulated for verification and rehearsal using an orthopedic surgical simulator.

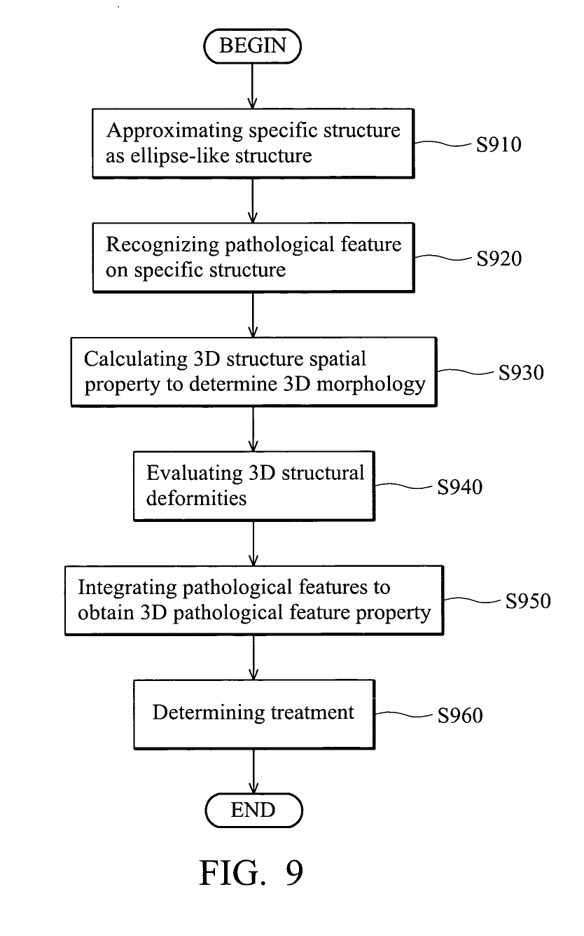

FIG. 9 is a flowchart showing an embodiment of an image analysis method for gleno-humeral joint morphology. In step S910, at least one specific structure is approximated as an elliptical (ellipse-like) structure at a plurality of transverse sections, where the elliptical specific structure can be evaluated on each transverse section using B-spline approximation. Additionally, the specific structure comprises the humeral stem, humeral head, contact joint, glenoid, or tubercle. It is understood that while the B-spline approximation is utilized in this embodiment, the invention is not limited thereto. In step S920, at least one pathological feature on the structure is recognized, and in step S930, at least one structural spatial property of a 3D structure is calculated based on the structural property of the elliptical structure, thus determining the morphology of the 3D structure. Additionally, the size or position of the pathological feature is further recognized, in which the concave, convex, and hole features are recognized as fractures, spurs, and tumors, respectively. Further, the morphology of the 3D structure comprises the axis, attitude, or center of the 3D structure. In step S940, structural deformities such as humeral head dislocation or compression of the 3D structure are evaluated according to the morphology of the 3D structure. In step S950, the pathological features on the sections are integrated to obtain at least one 3D pathological feature property, and in step S960, a treatment is determined accordingly. Note that details of the steps are discussed later.

Two-Dimensional Structure and Feature Recognition on Respective Transverse Sections

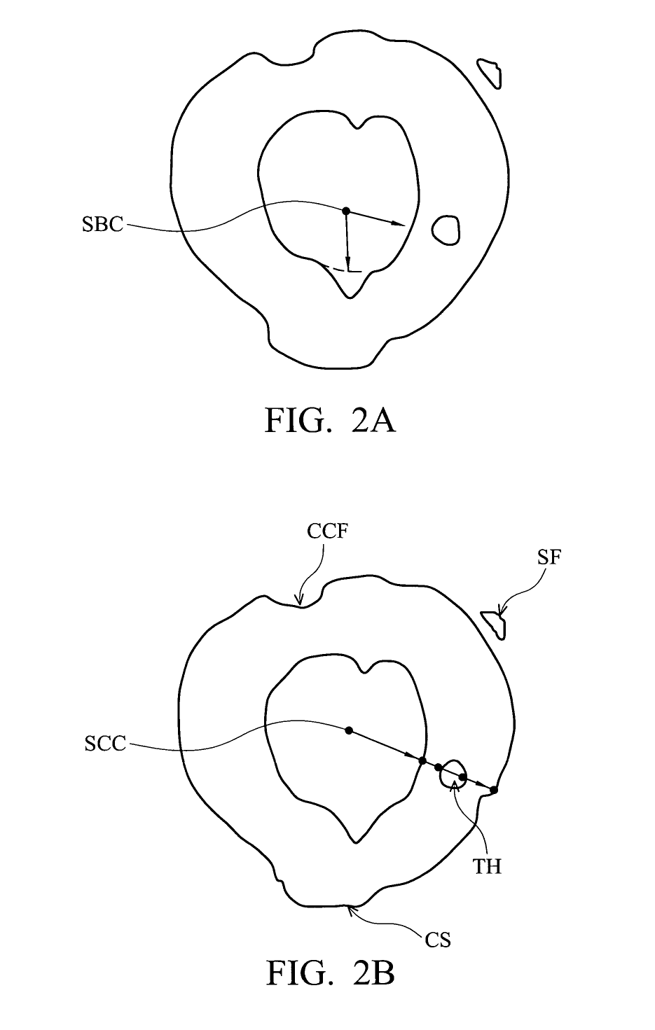

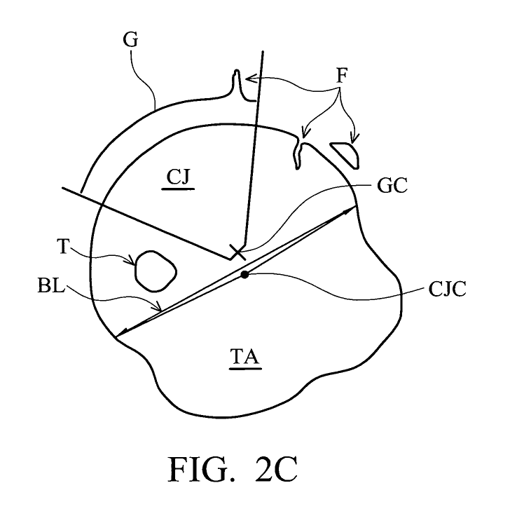

The recognition of elliptical structures and associated features is provided. The (initial) center of each stem canal on a transverse section is determined by averaging the positions of the pixels of the stem bone. A vector starting from the center (SBC) along every (totally 360) integral angular position is used to intersect the first bone (canal) boundary (FIG. 2A). Because the canal boundary is elliptical, the distance (radius (r)) from the center to the boundary changes smoothly except with concave and convex features. The radius inside a feature is interpolated by the two radii of the two feature ends. Then, 360 radii are used to re-determine the stem center using the B-spline approximation. Concave features such as concave fractures or convex features such as convex spurs on the bone boundaries, and hole (bone osteolytic lesion) features such as tumor holes (TH) inside the bone, or separate fractures (SF) outside the bone boundaries are then determined by changing the radius (r) from the stem center (SCC) to the bone boundaries as illustrated in FIG. 2B.

At the humeral head, the stem canal becomes obscure due to filling with cancellate bone. Therefore, a 2D humeral head center at each superior section resolving the humeral head is extrapolated by the stem canal centers at the inferior sections. A vector starting from this humeral head center along every integral angular position is then used to intersect bone boundaries in a manner similar to that described.

The concave and separate features are recognized as fractures (F), the hole features are tumors (T), the small convex features are spurs, and the arc (at the first intersected bone boundary) with a smooth radius change is recognized as the contact joint (CJ) as illustrated in FIG. 2C. The tubercle area (TA) is obtained by excluding the contact joint area, and the line connecting the boundary pixels of the two areas is defined as the boundary line (BL). The pixels on the contact joint are used to B-spline approximate the 2D contact joint center on the section. This center is used to re-determine the features at the contact joint area and the radii from this center to the pixels on the contact joint arc. The average of the radii of all the contact joint pixels is defined as the 2D contact joint radius on the transverse section. The intersections (at the second intersected bone boundary) with smooth radius changes at the lateral side are the pixels on the glenoid (G). These pixels are used to determine the glenoid center (GC) and average radius. This center is used to determine the concave fracture or convex spur on the glenoid.

Three-Dimensional Structure and Feature Property Calculations

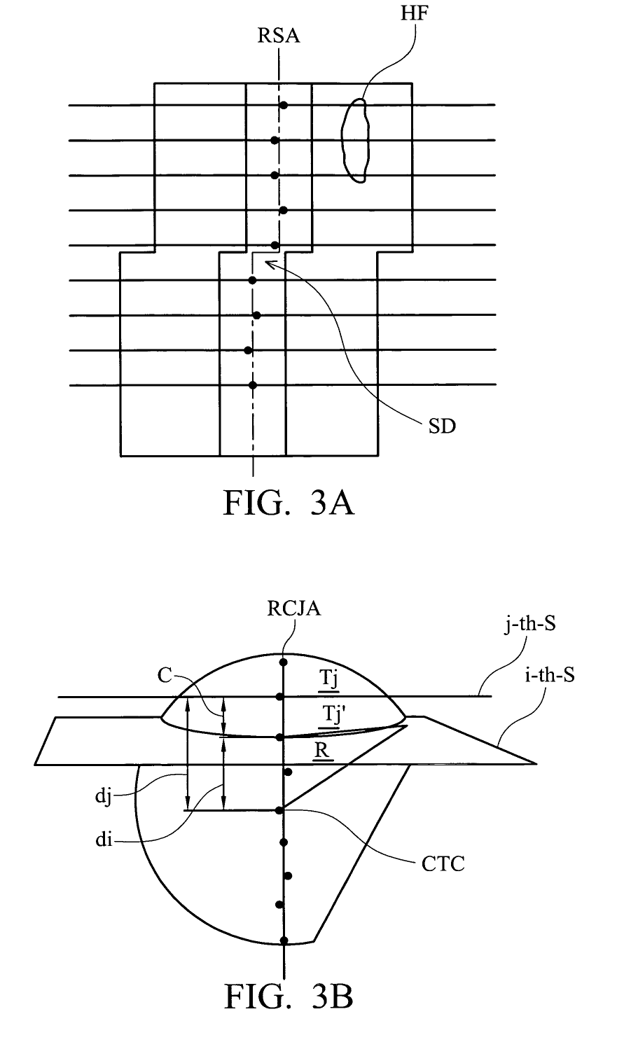

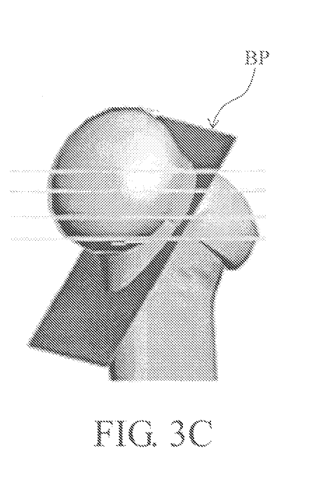

The 2D canal centers at the inferior sections are used to regress the stem axis (RSA). The 2D features at these sections are integrated to calculate the 3D position and volume for each 3D pathological feature such as hole feature (HF) as illustrated in FIG. 3A. If the distance between the centers of two neighboring sections is too large, a shear dislocation (SD) is considered to exist inside the stem. Two respective centerlines are used to regress the two sheared parts of the stem. To calculate the 3D contact joint center, the 2D contact joint centers obtained from superior sections are used to regress a contact joint axis. The 2D centers are then regressed onto this axis. The following formula by the Pythagoras theorem is used to determine the 3D center (FIG. 3B).

di²+ri²=dj²+rj²=R²ri²−rj²=dj²−di²=c(2di−c).

R is the 3D contact joint radius and assumed uniform. ri and rj are the 2D average radii at the i-th (i-th-S) section and the j-th (j-th-S) section, respectively. di and dj are the distances from the 3D contact joint center to the 2D center at the ith section and the j-th section, respectively. c is the interval between the two sections. The unknown di can be solved by c, ri and rj to determine the 3D center. One solution of the 3D contact joint center can be obtained from the most superior section with each of the other sections resolving the contact joint. The average of all the solutions is set as the 3D center. From this 3D center, the radius is determined as the average of the radii from the 3D center to all pixels on the contact joint.

The 3D glenoid center is determined by the method described. The glenoid attitude vector is then determined by averaging the vectors from the 3D center to all the pixels on the glenoid. The normal position of the contact joint center is then set as the addition of the 3D contact joint radius with the normal gap between the contact joint and the glenoid along the glenoid attitude vector.

The boundary plane (BP) (ax+by+cz=d) between the tubercle and the contact joint is approximated by regressing the boundary lines between the tubercle and the contact joint areas on the superior transverse sections.

$${A}^{T} AW = 0 , A = [ \begin{matrix}{X}_{1} & {Y}_{1} & {Z}_{1} \\ {X}_{2} & {Y}_{2} & {Z}_{2} \\ {X}_{3} & {Y}_{3} & {Z}_{3} \\ {X}_{4} & {Y}_{4} & {Z}_{4}\end{matrix} ] , \\ W = [ \begin{matrix}a \\ b \\ c\end{matrix} ] .$$

(X1, Y1, Z1), (X2, Y2, Z2), (X3, Y3, Z3), (X4, Y4, Z4)) are the boundary lines on the sections (FIG. 3C). One value for the constant d is then determined by one boundary line end at a superior transverse section. The average of all the values from the sections is assigned as d. Treatments for GH joint diseases are categorized into conservative treatments, open reduction, dissection and bone grafting, and arthroplasty. Conservative treatments are applied in the presence of only small spurs (convex features), fractures (concave or separate features), tumors (hole features) and humeral head dislocation.

Open reduction uses screws and plate or nails to fix a humerus or a glenoid with fractures. These fractures may also result in a humeral dislocation causing contact insufficiency of the humeral head with the glenoid. Morphological changes including bone fractures, humeral head or glenoid compression and humeral head dislocation are corrected during the open reduction. Dissection and bone grafting is used to remove large tumors. Screws and plate or nails may accompany to the open reductions and the bone grafting to fix the reduced fractured bone segments or the grafted bone.

Arthroplasty is applied in the presence of a large fracture, complex fracture, or tumor, or Avascular Neurosis (AVN) changing the contact joint radius irregularly or becoming much small at the humeral head. The prosthetic contact joint and the glenoid are set as the size at the normal shoulder. The radius of the prosthetic stem refers to the smallest radius of the stem canal from all the transverse sections to meet the requirement of the proximal cortical fit. The boundary plane between the tubercle area and the contact joint is set as the cutting plane to insert the prosthesis.

Surgical procedures of the above dissection and graft, open reduction and arthroplasty can be simulated to confirm suitability of the planned surgical procedures. All sizes of template prostheses are rendered in 3DS MAX representations. Each prosthesis is then converted into the volume representation to simulate the managed modality and procedures.

The results of three cases are introduced, where one is screw and T-plate, one is screw and washer, and one is arthroplasty. The intervals here were set as all in 3 mm for comparisons.

Open Reduction with Screw and T-Plate for Fractured Humeral Head

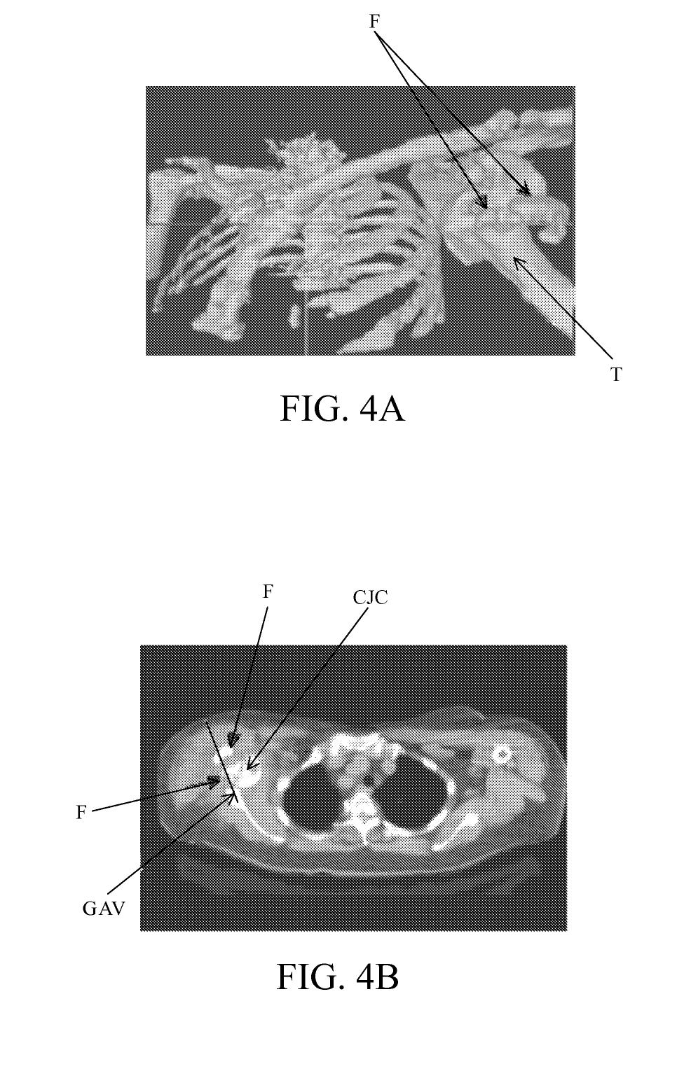

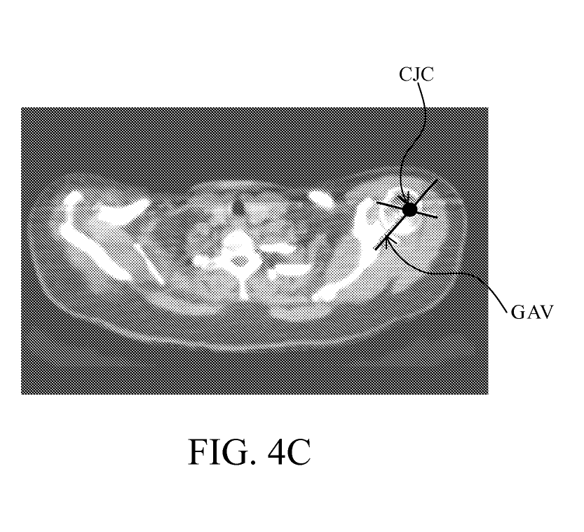

This case was performed in 54 CT transverse sections. FIG. 4A shows a 3D image rendered from the isosurface reconstructed from the MC isosurface reconstruction algorithm. At the left humeral head, two brought outward head fractures (F) and the head dislocation (HD) can be clearly observed. FIG. 4B shows a transverse section resolving the right humeral stem and the left humeral head and the image analysis result for this section. The result at the right humeral stem demonstrates a B-spine approximated canal without pathological convex, concave and hole features. The result at the left humeral head shows the two head fractures (F), the contact joint center (CJC) and the glenoid attitude vector (GAV) that reveals a large deviation (dislocation) from the contact joint center to the glenoid attitude vector. FIG. 4C shows a transverse section resolving the normal right humeral head and glenoid at which only little deviation from the contact joint center to the glenoid attitude vector can be observed.

The calculated deviations of the humeral heads to the ideal positions are (−1.3, −1.0, 1.5) and (9.1, −7.8, 16.5) for the right and left shoulder respectively. These indicate a left humeral dislocation (about 20 mm). Table 1 shows image analysis results of the transverse sections resolving the humeral head fractures and dislocation. In each section, two concave fractures exist on the left head boundary, and the distance between the left 2D contact joint center and the ideal position is large, revealing the dislocation and fracture at the left humeral head. The angular positions of each fracture in the consecutive sections are close to each other, indicating the 2D fractures are the same 3D fracture. The 2D fractures are integrated to obtain the 3D fracture position and volume.

TABLE 1

| | | | | | Right | Left | | Left | | |
|---|---|---|---|---|---|---|---|---|---|---|
| | | | Right | Left | contact | contact | Right | glenoid | | |
| Section | Fracture 1 | Fracture 2 | contact joint | contact joint | joint | joint | glenoid | center | Right | Left |
| No. | (andles) | (andles) | center (x, y, z) | center (x, y, z) | radius | radiu | center (x, y, z) | (x, y, z) | gap | gap |
| 26 | 143~176 | | | (102.2, | | 18.6 | | | | 20.7 |
| | | | | 220.8, 0) | | | | | | |
| 27 | 135~176 | 98~117 | | (100.9, | | 20.4 | | | | 13.3 |
| | | | | 219.1, 0) | | | | | | |
| 28 | 112~172 | 97~111 | | (102.3, | | 19.9 | | | | 11.4 |
| | | | | 218.6, 0) | | | | | | |
| 29 | 117~165 | 98~111 | | (102.2, | | 19.2 | | | | 9.6 |
| | | | | 219.4, 0) | | | | | | |
| 30 | 129~188 | 121~165 | | (101.3, | | 18.8 | | | | 4.2 |
| | | | | 219.4, 0) | | | | | | |
| 31 | 125~161 | | | (102.1, | | 17.1 | | | | |
| | | | | 219.2, 0) | | | | | | |
| 32 | | | | (101.0, | | 15.6 | | | | |
| | | | | 218.7, 0) | | | | | | |
| 33 | | | | (100.9, | | 14.7 | | (50.9, 170.3, | | |
| | | | | 217.8, 0) | | | | 0) | | |
| 34 | | | | (99.4, 219.4, | | 10.1 | | (50.9, 170.3, | | |
| | | | | 0) | | | | 0) | | |
| 35 | | | | | | | | (62.3, 193.0, | | |
| | | | | | | | | 0) | | |
| 36 | | | | | | | | (50.0, 180.8, | | |
| | | | | | | | | 0) | | |
| 37 | | | | | | | | (59.8, 194.5, | | |
| | | | | | | | | 0) | | |
| 38 | | | | | | | | (45.9, 180.9, | | |
| | | | | | | | | 0) | | |
| 39 | | | | | | | | | 4.1 | |
| 40 | | | (385.5, | | 21.5 | | (402.8, | | 3.5 | |
| | | | 203.7, 0) | | | | 175.9, 0) | | | |
| 41 | | | (383.4, | | 19.3 | | (400.9, | | 4.1 | |
| | | | 206.3, 0) | | | | 175.9, 0) | | | |
| 42 | | | (383.6, | | 20.0 | | (402.2, | | 4.2 | |
| | | | 207.4, 0) | | | | 182.8, 0) | | | |
| 43 | | | (381.6, | | 19.8 | | (412.5, | | 4.2 | |
| | | | 206.3, 0) | | | | 172.5, 0) | | | |
| 44 | | | (382.5, | | 20.7 | | (409.3, | | 4.4 | |
| | | | 204.6, 0) | | | | 180.5, 0) | | | |
| 45 | | | (384.7, | | 22.9 | | (415.3, | | 4.3 | |
| | | | 202.7, 0) | | | | 177.2, 0) | | | |
| 46 | | | (382.2, | | 18.8 | | (411.6, | | 4.6 | |
| | | | 206.0, 0) | | | | 180.9, 0) | | | |
| 47 | | | (385.6, | | 18.4 | | | | 5.3 | |
| | | | 205.8, 0) | | | | | | | |

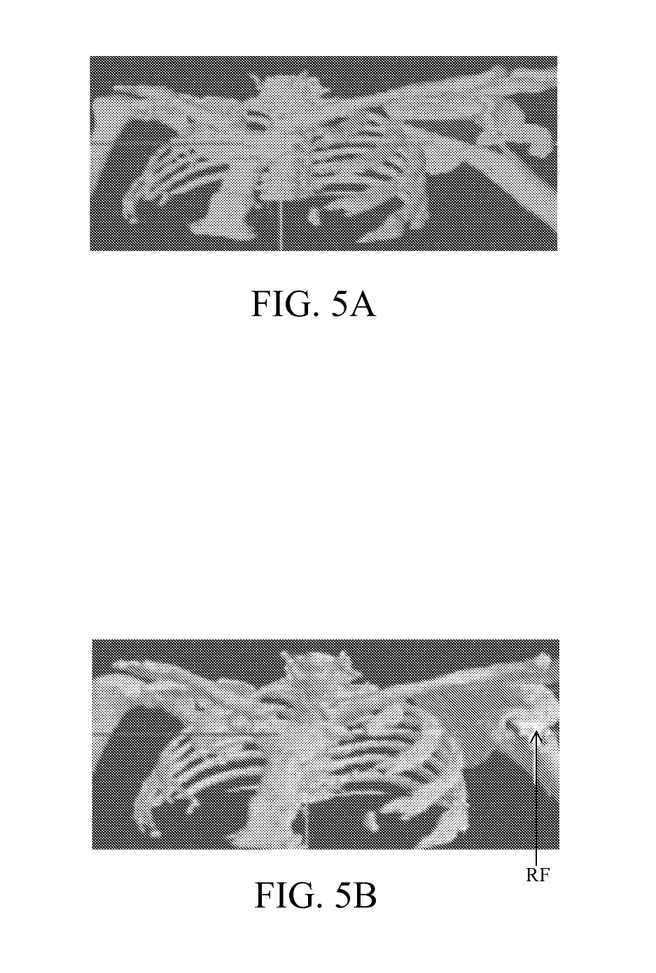

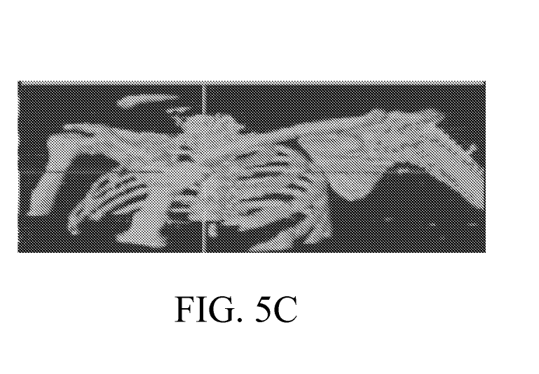

FIGS. 5A, 5B and 5C show simulated images for the open-reduction surgery. FIG. 5A shows a tool swept surface being cut the first tuned out bone fragment. FIG. 5B shows the tuned out fragment repositioned onto the head in a translation following a 90-degree rotation. FIG. 5C shows the left humeral head recognized and repositioned near the glenoid, where the dislocation is reduced, and the calculated screw and T-plate (sizes and positions) are used to fix the repositioned fractured fragment onto the humeral head. Because the repositionings of the two fractured fragments and the humeral head are in accordance with the calculations of the system, the reductions are suitable.

Arthroplasty for Lumeral Head with Tumor and Communicate Fractures

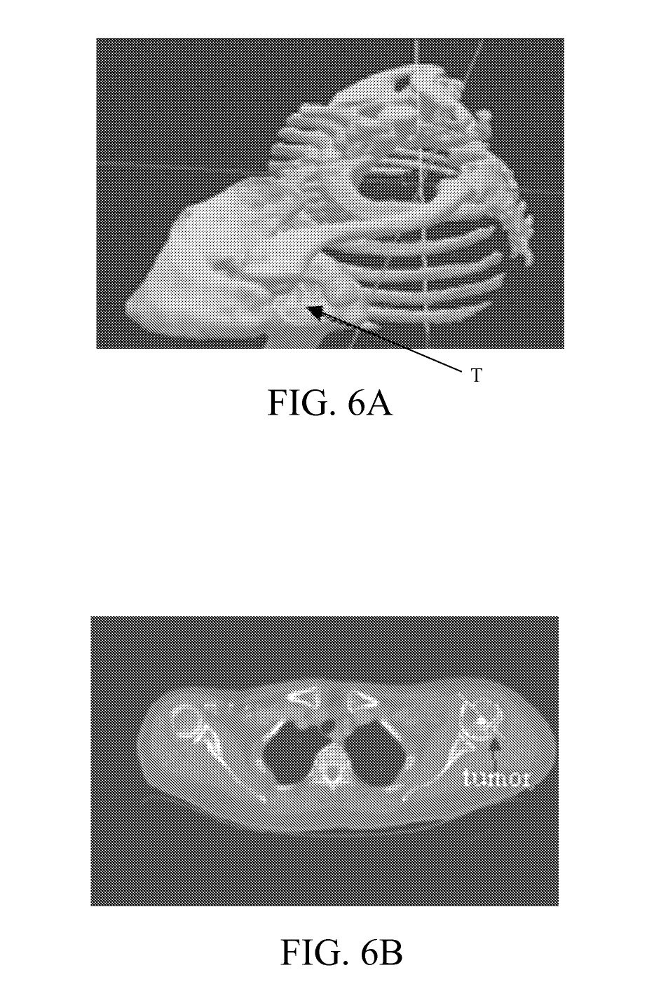

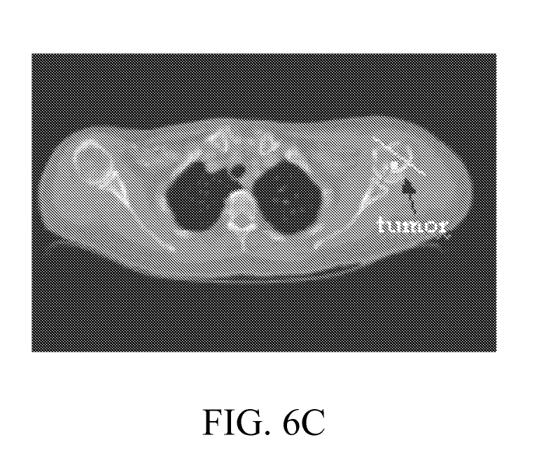

This case was performed in 67 CT transverse sections. FIG. 6A shows a 3D reconstructed image of a tumor on the right humeral head. FIG. 6B and FIG. 6C show respective transverse sections resolving a respective hole generated by the same tumor hole. Because the outside cortical bone is only slightly fractured on the lateral side, this pathology is observed from a specific perspective (FIG. 6A). However, this pathology hole can be clearly analyzed at the superior transverse sections to indicate the tumor position and volume.

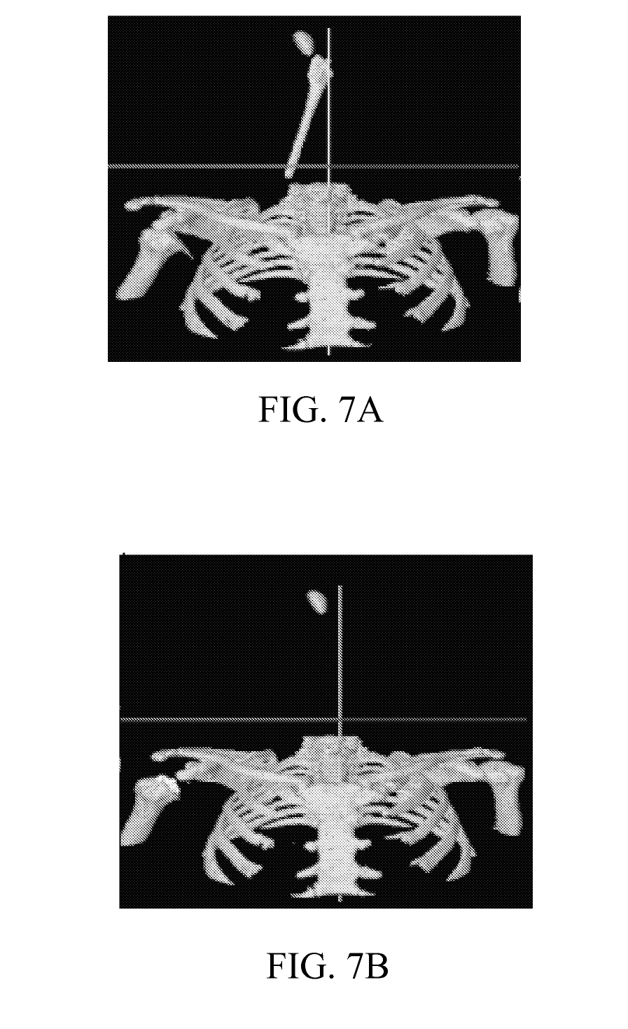

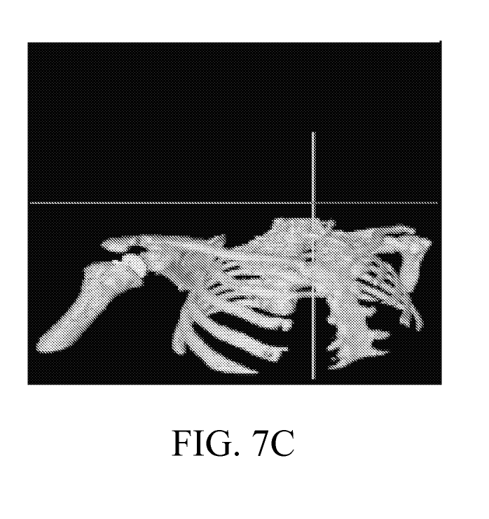

The image analysis result indicates one large hole tumor (with large radius change) in each section resolving the superior right humeral head. Because of this tumor, artroplasty is required. The calculated parts corresponding to the simulation of the arthroplasty are performed based on feature recognition, evaluation and surgical management. FIG. 7A shows a saw cutting the humerus along the boundary plane between the tubercle and the contact joint. The calculated plane is used to insert the prosthetic head. FIG. 7A also shows the calculated prosthetic stem and head.

FIG. 7B shows the recognition and insertion of the prosthetic stem. Before this insertion, the humerus is recognized and translated to ease the surgical simulation. FIG. 7C shows the insertion (following recognition) of the prosthetic head, and the movement of the humerus into a normal location. The position and size of the prosthesis result in good morphology matching with the normal left shoulder. These arthroplasty simulations indicate the planned arthroplasty surgery can result in good shoulder function postoperatively.

Open Reduction with Screw and Washer for Avulsion Fracture

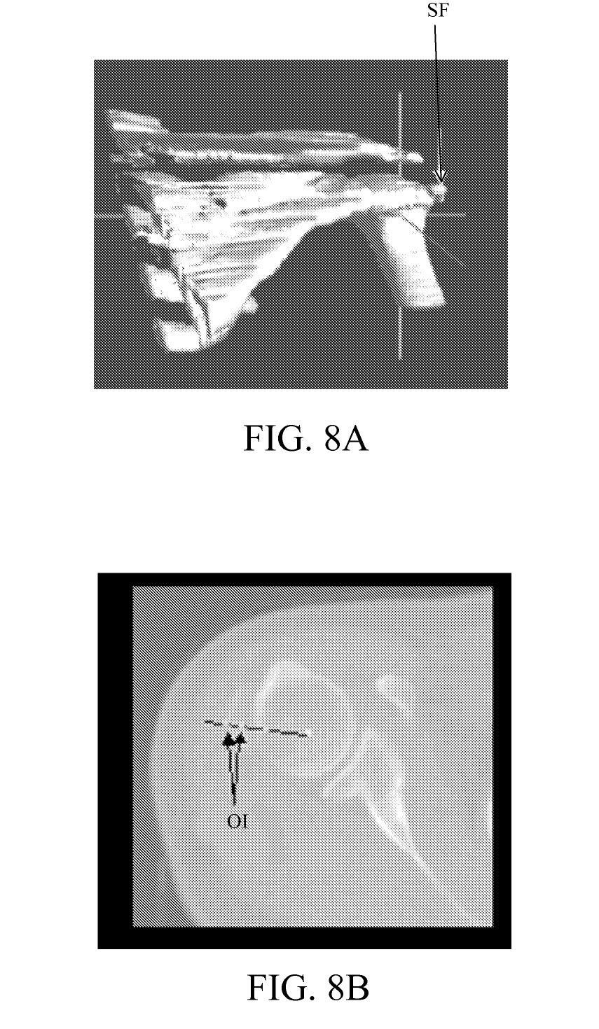

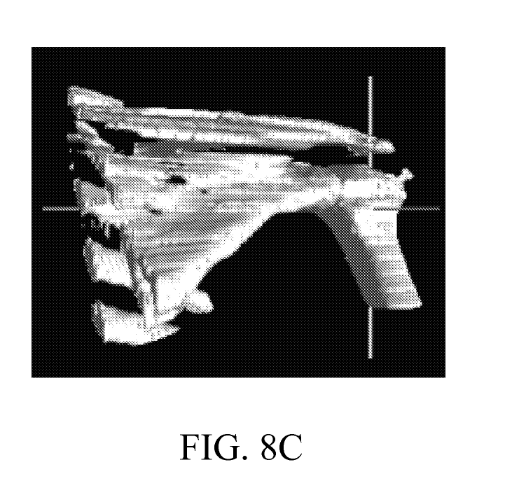

This case was performed in 49 CT transverse sections. FIG. 8A shows a 3D reconstructed image demonstrating the separated fracture outside the right humeral head. FIG. 8B shows a transverse section resolving the right humeral head with the separated fracture. FIG. 8C shows the surgery simulation result, where the separate fragment is recognized and repositioned back onto the humeral head, and a screw and washer are positioned the separate fragment.

In the invention, the 3D geometry of humerus and glenoid bones is analyzed to estimate the humeral head dislocation, and fractures and tumors in the humerus and glenoid. As a result, precise diagnoses and surgical procedures for tumor dissection and bone graft, open reduction and artroplasty can be automatically determined.

Image analysis methods for gleno-humeral joint morphology, or certain aspects or portions thereof, may take the form of program code (i.e., executable instructions) embodied in tangible media, such as floppy diskettes, CD-ROMS, hard drives, or any other machine-readable storage medium, wherein, when the program code is loaded into and executed by a machine, such as a computer, the machine thereby becomes an apparatus for practicing the methods. The methods may also be embodied in the form of program code transmitted over some transmission medium, such as electrical wiring or cabling, through fiber optics, or via any other form of transmission, wherein, when the program code is received and loaded into and executed by a machine, such as a computer, the machine becomes an apparatus for practicing the disclosed methods. When implemented on a general-purpose processor, the program code combines with the processor to provide a unique apparatus that operates analogously to application specific logic circuits.

While the invention has been described by way of example and in terms of preferred embodiment, it is to be understood that the invention is not limited thereto. Those who are skilled in this technology can still make various alterations and modifications without departing from the scope and spirit of this invention. Therefore, the scope of the present invention shall be defined and protected by the following claims and their equivalents.

What is claimed is:

1. An image analysis method for gleno-humeral joint morphology, comprising:
providing a plurality of transverse sections;
approximating at least one specific structure as an elliptical structure at each of the transverse sections;
recognizing at least one pathological feature on the specific structure;
calculating at least one structural spatial property of a 3D structure based on at least one structural property of the elliptical structure, thus determining the morphology of the 3D structure; and
evaluating structural deformities according to the morphology of the 3D structure.

2. The method of claim 1 further comprising approximating the specific structure as the elliptical structure using B-spline approximation.

3. The method of claim 1 wherein the specific structure comprises a humeral stem, humeral head, contact joint, glenoid or tubercle.

4. The method of claim 1 further comprising recognizing the size or position of the pathological feature on the specific structure.

5. The method of claim 1 further comprising recognizing a concave feature on the specific structure as a fracture.

6. The method of claim 1 further comprising recognizing a convex feature on the specific structure as a spur.

7. The method of claim 1 further comprising recognizing a hole feature on the specific structure as a tumor.

8. The method of claim 1 wherein the morphology comprises an axis, attitude, or center of the 3D structure.

9. The method of claim 1 wherein the structural deformities of the 3D structure comprise humeral dislocation or compression.

10. The method of claim 1 wherein the structural property of the elliptical structure comprises the center or radius of the elliptical structure.

11. The method of claim 1 wherein the structural spatial property comprises a center or radius of a contact joint, the attitude of a glenoid, boundary planes between a contact joint and a tubercle, or a stem axis.

12. The method of claim 1 further comprising:
integrating the at least one pathological feature on the sections to obtain at least one 3D pathological feature property; and
determining a treatment according to the 3D structure and the 3D pathological feature property.

13. The method of claim 12 wherein the treatment comprises a conservative treatment, open reduction, dissection and bone grafting, or arthroplasty.

14. The method of claim 12 further comprising determining surgery parameters according to the 3D structure and the 3D pathological feature property.

15. The method of claim 14 wherein the surgery parameters comprise a reduced distance between a dislocated humerus and fracture fragments, volume and position for tumors dissection and bone grafts, or size and position of a prosthesis or screws and plate.
